(19) United States
(12) Patent Application Publication
(10) Pub. No.: US 2003/0064362 A1
(43) Pub. Date: Apr. 3, 2003

(54) Biosensor for detection of toxic substances

(76) Inventor: Robert B. Silver, Grosse Pointe, MI (US)

Correspondence Address: DONALD J. SILVERT, 637 MICHELLINE LANE, NORTHBROOK, IL, 60062, US

(21) Appl. No.: 10/259,026

(22) Filed: Sep. 27, 2002

Related U.S. Application Data
(60) Provisional application No. 60/325,850, filed on Sep. 28, 2001.

(51) Int. Cl. C12Q 1/70; G01N 33/53; G01N 33/567; G01N 33/554; G01N 33/569; G01N 33/543

(52) U.S. Cl. 435/5; 435/7.1; 435/7.32; 436/518; 435/7.21

(57) ABSTRACT

The present invention relates to a biosensor, and in particular, to a biosensor for comprehensively detecting toxic substances. The invention uses biological cells that have been bio-engineered to detect both particular species of toxic substances as well as classes thereof. Methods of using the biosensor so described are set forth as well.

This application incorporates in its entirety and claims benefit of U.S. Provisional Application No. 60/325,850, filed Sep. 28, 2001.

The invention relates to a biosensor, and in particular, to a biosensor for comprehensively detecting toxic substances, such as substances developed for the purpose of impairing or killing living matter as well as those developed for legitimate civilian use but nevertheless has toxic effects if presented in sufficient concentration.

Chemical and biological agents abound that have toxic effects if sufficient concentrations thereof are directed at humans and other living organisms. While most such agents either occur in nature and exist generally in sufficiently low concentration as to not pose a danger or were developed for appropriate civilian use and are typically safe if used in accordance with manufacturer's instructions, it is the case that naturally occurring biological agents as well as chemical agents can be manufactured and applied purposely or by accident in concentrations that would be highly detrimental to a civilian population or to military units in action or otherwise.

Exemplary chemical or biological threats include: Lethal Factor, S-layer components (e.g., EA1), Protective Antigen and Edema Factor of B. anthracis; Yersinia pestis; Staph enterotoxin A or B or TSST; Botulinum toxins, Vibrio sp.; hemorrhagic viruses (e.g., hantavirus, arenavirus, Ebola); and genetically altered variants of such biotoxic agents.

What is needed is a sensor that is implacable and/or man-, animal- or semiautonomous- or autonomous-vehicle portable, and that provides rapid warnings, including detailed analytical data, that can stand alone or complement other analytical data obtained by other systems. No broad spectrum sensor having such characteristics is currently available, and is certainly needed.

### SUMMARY OF THE INVENTION

It is an object of the present invention to provide materials useful in the detection of toxic substances.

It is a further object of the present invention that such materials are packaged in a unit that can be issued to appropriate personnel, or mounted on vehicles or at stationary sites for portable or remote sensing of toxic substances.

It is yet a further object of the present invention that the unit detect toxic substances broadly and rapidly, with acceptably low to no false positives or negatives, such as might be imposed by conformers, enantiomers, or chimeric agents.

Another object of the present invention is to provide assessment of biosensor activity at the biosensor site and/or at a remote location using various communication means, such as lighted labels, LED patterns, tactile or other stimulators of physiological senses, using open or secure coded messages, that may be transmitted to a remote location for evaluation by human and/or machine experts, Le., using expert agent technology, in part as described in the MATRRIKS system set forth in U.S. Ser. No. 08/618,246.

Yet another object of the present invention is to provide a means of subsequent collection and detailed evaluation of the cause of triggering the biosensor.

Another object of the present invention is that it have low maintenance requirements and long operational life, be readily configured for changing customer requirements, be compatible with other detection systems, and the like.

### DETAILED DESCRIPTION OF THE INVENTION

The present invention is predicated on use of biological cells that are capable of reacting to physical or chemical deleterious effects, such as, for example, effects of electromagnetic forces, nuclear energy, toxic chemicals, or pathogens (collectively referred to herein as “Foreign Substances”). Preferred such cells include animal cells having the specialized function of identifying foreign matter with respect to the animal, such as detection of foreign cells and viruses as occurs in bacterial and viral disease conditions. More preferred, the cells used in the context of the present invention include any class of macrophage. Yet more preferred, the cells used are conventional or circulating or stationary macrophages. Most preferred cells are stationary macrophages. Especially most preferred such cells are of a hitherto unidentified class of stationary macrophages found in small canals or ducts within dense irregular connective tissue of an animal. As demonstrated herein below, the materials and method of the present invention capitalizes on the characteristics of these macrophages to react to foreign materials, such that they can be harnessed as sensory elements, whether in unmodified or modified form.

Single cells, as units of living organisms, are the targets of the destructive effects of Foreign Substances. While the effects of most Foreign Substances are focused to selective molecules and classes of molecules, several characteristic effects of Foreign Substances on cells are common among cells, most notable among these are increases in intracellular concentration of “free” calcium and free radicals. Indeed the responses of cells to exogenous stimuli are usually expressed through calcium signals or free radicals. Several cell types serve as sentinels that respond to dangerous compounds via predictable and aggressive actions, e.g., macrophages, T-cells and sensory neurons and epithelia. Single cells embody the full range of sensitivities to Foreign Substances, and harnessing the inherent means of signaling that exposure to Foreign Substances is the subject of the present invention. Indeed, because cells possess a substantial breadth of potential sensitivities to Foreign Substances, including those yet to be identified or hurled against living cells, the present invention stands to provide substantial information to help in disease control and chemical and biological warfare scenarios.

The means of monitoring cell signals are known and presently in use in basic research studies of command and control of cell functions. Using protocols known in the art provides suitable dosimetry for classes of Foreign Substances.

Preferred macrophages used in the context of the present invention are derived from cold-blooded (Le., poikilothermic) organisms. Preferred such poikilotherms include, but are not limited to, echinoderms (e.g., sea urchins, star fish, sand dollars, and the like), teleost fishes (e.g., Opsanus tau), elasmobranchs (e.g., skates, rays), mollusks (e.g., squid, cuttlefish, and the like), amphibians, or other animals. In addition, warm-blooded animals are also a useful source of the macrophages used in the present invention, such as, for example, birds. Most preferred such macrophages used in the context of the present invention are isolated from the wall of the vestibular semicircular canals of oyster toadfish or their structural and functional analogues.

Preferred macrophages have characteristics whereby they respond to bacterial surface antigens, viruses, prions, bio-active substances and particulate samples (all of which are included in the term Foreign Substance, defined herein above) at suitably fast rates after initial exposure thereto. The characteristic response of preferred macrophages relates to full activation and initiation of phagocytic activity, and primary reactions that are preliminary to such activity, as are well known in the field. See Vadivelooo, J. Leukocyte Biology 66:579-582 (1999); Gijon and Leslie, J. Leukocyte Biology 65:330-336 (1999); Chow et al., New Horizons 3:342-351 (1995); Jaconi et al., Eur. J. Pediatrics 152:S26-S32 (1993); Adams, Immunology Today 10:33-35 (1989); Wilkinson, Recent Results in Cancer Research 56:41-48 (1976). A suitable rate for the phagocytic effect is less than about 90 seconds; more preferably, less than about 60 seconds from first exposure. A suitable rate for the primary reactions preliminary to the phagocytic effect is less than about ten seconds, more preferably less than about five seconds, yet more preferably less than about one second of the binding or effect of the Foreign Substance.

A particularly preferred macrophage used in the context of the present invention includes a reporter system incorporated therein. Such a reporter system is one that has been preferably genetically engineered into the macrophage. A preferred reporter system is any that can be induced to impart a signal upon activation of the macrophage in response to contact with a Foreign Substance, such as, for example, a luminescent or fluorescent reporter system. Preferred luminescent reporter systems include those that use aequorin, obelin, luciferase and the like. Preferred fluorescent reporter systems include those that use fura2 and the like.

In one embodiment, the present invention relates to a method for detecting a Foreign Substance in a fluid, comprising contacting a first macrophage having a reporter system incorporated therein with said fluid. The Foreign Substance can be any substance or radiation that is not native to the macrophage used in the context of the present invention, which substance or radiation is either toxic or not toxic to the macrophage. In a preferred embodiment, the Foreign Substance is toxic. In particular, the Foreign Substance is an allergen, a pathogen or a toxin, wherein the pathogen is selected from the group consisting of a bacterium, a prion, or a virus, and wherein the toxin is a chemical agent having toxic effect on a living cell, such as being an inhibitor of a metabolic pathway. Such chemical agents are bioactive agents that can be inorganic or organic compounds, and are preferably small organic compounds having a molecular weight that generally does not exceed about 2,500 daltons. Typically, the radiation-variety Foreign Substance that can be detected using the present invention will have detrimental impact on a living cell, and includes, for example, x-ray, alpha and gamma radiation. Allergens can be pathogens or toxins as well, but is preferably used herein to relate to pollen, dust and other typically sub-toxic substances that commonly induce an allergic reaction.

In particular, the first macrophage has been treated in a fashion as set forth herein below to identify a particular Foreign Substance. When it is desirable to identify more than one Foreign Substance, then a second macrophage, appropriately treated, is employed. The macrophage is isolated from the body of the host organism, or may be obtained from a suitable cell culture or stem sells of established primary or immortalized cell line. In a preferred embodiment, the macrophage is isolated from the dense irregular connective tissue of the semicircular canal of the inner ear. The isolated cell is then cultured in culture media under conditions that are known to the art.

The present invention requires contact to be made between the macrophage as set forth herein above and a Foreign Substance, which is presented to the macrophage via a fluid. Preferred fluids are ambient air and a liquid, particularly water.

The combination of a macrophage used in the context of the present invention and a fluid containing a Foreign Substance is that the reporter system is preferably triggered. The triggering of the reporter system preferably is registered by an electronic mechanism.

The present invention in another embodiment also relates to the use of a suitable macrophage having a reporter system incorporated therein for detecting a Foreign Substance. A suitable macrophage can be derived from any animal, and is preferably derived from a poikilothermic animal or cell. Preferably, the reporter system incorporated into the macrophage of the present invention is luminescent or fluorescent, such as, for example, aequorin, obelin, or fura2, Quin2, calcium green, calcium orange, and the like, including, but not limited to, the acetoxy-methyl esters. The macrophage preferably used is conventional or stationary, and is more preferably stationary.

The present invention preferably couples the reaction of the aforementioned macrophage upon its exposure to a Foreign Substance to a suitable reporter system so that the occurrence of presentation of the Foreign Substance to the macrophage can be readily communicated. One embodiment of the present invention involves use of the large amplitude calcium signal that occurs upon activation of the macrophage, which calcium signal can be identified by intracellular combination of the calcium with, for example, a luminescent reporter system (e.g., aequorin, obelin, and the like) or a fluorescent reporter system (e.g., fura2, Quin2, calcium green, calcium orange, and the like).

As noted above, the macrophages used in the context of the present invention preferably are bio-engineered to respond to particular Foreign Substances. One approach to such bio-engineering is to obtain the DNA sequence that encodes the cell surface receptors for said Foreign Substance of interest, using standard methods known in the art for obtaining such DNA sequences. Such efforts include immunizing mice with the Foreign Substance of interest, which, preferably, is selected from the group consisting of bacteria, pollens, viruses, prions, and the like. Foreign Substances of interest also include metabolic toxins and narcotic precursors and by-products. A more expanded listing of Foreign Substances of interest includes, for example, Anthrax, Bioregulators, Botulinum toxin, Brucellosis, Cholera, Clostridium perfringens, Encephalomyelitis viruses, Glanders, Hemorrhagic fever virus, Marine toxins, Mycotoxins, Plague, Q fever, Ricin toxin, Shigellosis, Smallpox virus, Staphylococcal enterotoxins, Tularemia, Typhus, Chimeras and Homologues of any of the identified Foreign Substances, Foot and Mouth, Fowl Pest, Hog Cholera, Rinderpest, Bean Blight, Corn Smut, Potato Late Blight, Wheat Stem Rust, Narcotic and non-narcotic drugs and precursors, and so-called nerve, blister and blood agents (e.g., GA, GB, Sarin, Vx and the like, used as chemical weapons) and their conjugates to carrier proteins such as ovalbumin (a practice well known to those schooled in the art of producing antibodies to haptens).

After a suitable number of days, as known and practiced in the art, the immunized mice are sacrificed or otherwise bled and T cell and B-cell populations are collected. T-cells and B-cells that are positive for the Foreign Agent(s) of interest are then used to create hybridoma cell lines using a standard protocol.

RNA populations that include sequences encoding B-cell surface antibodies of interest are prepared from the Foreign Agent-positive cells, again using standard protocols known in the art. RNA species specific for the cell surface antibodies that recognize the Foreign Substance of interest are isolated and used to generate a genetic construct that can be used to transform a macrophage cell.

As noted above, the macrophage used in the present invention is preferably a stationary macrophage, more preferably an immortalized stationary macrophage, which has been immortalized using standard protocols known in the art, such as, for example, with HTLV-1 or myeloma fusion.

The stationary macrophage is preferably transformed with the genetic construct that encodes the aforementioned cell surface antibody. Transformed stationary macrophages having particular utility in the context of the present invention can be selected by subjecting the transformed cells to the Foreign Substance of interest and scoring the cells for calcium flux, using standard methods known to the art.

Alternative genetic constructs used to transform stationary macrophage can have multiple chimera receptors (e.g., macrophage intra-membrane and cytoplasmic domains of cytokine receptor of the stationary macrophage and extracellular and intra-membrane ligand binding domains of educated B-cell). For methods that are employed in generating the cells of the present invention, see, inter alia, Chang and Clevenger, Molec. and Cellular Biology, 18:896-905 (1998); Chaika, et al., J. Biol. Chem., 272:11968-11974 (1997); Boehm, et al., J. Biol. Chem. 273:7169-7176 (1998). Accordingly, lines of transformed macrophage cells can be established that recognize specific Foreign Substances or classes of Foreign Substances for those Foreign Substances that have a chemical nature.

Lines of macrophages or other cells can also be established that react to Foreign Substances that have a physical nature only, such as for example, nuclear radiation or other deleterious electromagnetic force.

Preferably, a detection device of the present invention includes culture cells in a multi-well culture device known in the art, wherein each well contains hydrogel, or some other suitable matrix that maintains cellular vitality, and culture media including a detection molecule, such as a lumiphore, for example. Alternative detection molecules are set forth above, and including labeling with fluorescent or absorptive probes for Foreign Substances of interest.

On binding of a Foreign Substance of interest, cells having the appropriate cell surface antibody that are included in the detection device of the present invention activate and emit photons from calcium-dependent luminescence, as known in the art. Pattern of luminescent wells versus controls, wherein each well contains a line of transformed or non-transformed cells that have known capability to bind to different Foreign Substances, is characteristic of a Foreign Substance of interest, which pattern is decoded by on-board logic and displayed through on-board display and/or transmitted to remote receiver and decoder.

After detection of an agent, cells can be retrieved and residual bodies probed for identity of activating agent. For example, T-cell and B-cell clones produced will be available for subsequent analysis of residual bodies. General metabolic toxins will also provide luminescent output, which will be classifiable due to broad array response.

Unknowns will be identifiable through activation of controls and suitable general class-identifier cells.

Common among a variety of cell activation processes is an increase in intracellular concentration of “free” calcium and free radicals. Indeed, the responses of cells to exogenous stimuli, such as Foreign Substances, are usually expressed through calcium signals or free radicals.

Several cell types serve as sentinels that respond to specific compounds via predictable and aggressive actions, e.g., macrophages, T-cells and sensory neurons, intestinal- and sensory epithelia, and free-radical sensing fungi. Similarly, numerous additional cell types respond to metabolic activation or redirection, programmed cell death (apoptosis), metabolic alterations, and cell disruption et cetera in characteristic manners. In each of these cases it is well known that such alterations in cellular state involves changes in the levels of “free” calcium and often free radical levels, as well. Such changes can be readily annunciated in vivo and in vitro. Such changes can be readily detected with specific optical reporter molecules whose photonic output is luminescent, fluorescent or absorptive, or a combination of those means.

Cells labeled with appropriate luminescent annunciators can be cultured in a small-volume culture vessel. The vessel serves as the cells' living environment, provides a means of entry for the Foreign Substances in question, and be coupled to an optical sensing device. Vessels will be clustered into arrays that can provide for both redundancy (several vessels with identical cell types) and selectivity (sets of vessels each with its own cell type tuned for a given Foreign Substance).

Cells exposed to a Foreign Substance will “activate” resulting in a rapid increase in specific reactions and general metabolic activity—e,g,, oxidative phosphorylation/intermediary metabolism, phagocytosis and motility. Such changes result in dramatic increases in calcium signals and free radical formation, among others. Those signal molecules react with available enunciators—e.g., aequorin, green fluorescent protein constructs, luminol—resulting in emission of photons. Photons are detected by photodiodes, avalanch photodiodes, single or multiple microchannel plates or CCDs (Charge-Coupled Devices) in contact with the transparent culture vessel—which also serves as a light conduit to the optical detector.

Suitable cells used in the context of the present invention are those that are typically quiescent and thus have intrinsically low basal metabolic demands—e.g., stationary macrophage, which are naturally occurring sentinels. Oocytes (such as those from surf clams Spicula solidissima) or other suitable quiescent, activatable cells may also be used. Depending upon the cell types used and cell demands regarding size, mass, power, temperature and culture conditions, such a sensor can remain on-station—awaiting a biochallenge—for periods that may range from less than 1 through 60 or more days.

Among the specialized cellular systems are those responsible for detection and discernment of conditions specific to the organism, in part or in whole. Systems are also present to limit the effects of significant agents, and to recover from insults by these agents. Methods for detecting discrete response signatures in calcium in several cell types are known in the art. See, e.g., Silver, “Imaging and measurement of intracellular free calcium in living cells,” in Cells: A Laboratory Manual, Spector, et al., editors, Cold Spring Harbor Laboratory Press, pp 80.1-13 (1997); Silver, Cell Calcium, 20:161-179 (1996); Silver, et al., Biol. Bulletin, 187:293-299 (1994); Llinas, et al., Science, 256:677-679 (1992). Similar reporters exist for other classes of messengers important for cellular signaling, e.g., free radicals, protons, adenosine triphosphate, et cetera. Thus, methodologies matured in basic research applications can be readily extended for detecting cellular responses to Foreign Substances in manners consistent with application of the present invention.

In practice, such a system preferably involves the genetic transformation of suitable cells with the appropriate luminescent probes for calcium, free radicals. Signals detected are composed of luminescent photonic output from activated cells, thus obviating a/the requisite requirement for input excitation illumination. Preferably, these cells are cultured in vessels that have an array of optical and electrical sensors, for signal detection, output and power input, and components permitting local and/or remote communication, read-out and analysis. Detected patterns can be analyzed with on-board computational resources. Cellular responses to significant agents can be seen as discernible, discrete signals in macrophages, T-cells and other detection cells. Both natural and preselected cells (e.g., those selected/engineered for their reaction with agents of interest) can be used as naive and educated sensors. The response spectrum to significant agent(s) preferably minimize false positives and negatives. The use of both high affinity and low affinity receptor for the agents of interest (i.e., to be detected) permit assessments of agents both known, and those which may be unknown, but have molecular physiognomy similar to that of known agents. This approach can also be used to detect molecules selective and/or specific for specific strains, species, genus, et cetera. When detailed identity of an agent(s) is needed, the detecting cells can be harvested from the detector device and analyzed by conventional methods, including solid-state immunoassay (e.g., immunofluorescence, immunoblot, so-called bio-chips) and analysis of residual bodies (e.g., macrophages) complexes. This approach permits assessment of multiple classes and specific agents, both known and previously unencountered in a system whose life support is sustainable in an unattended field application for periods of about 4 to at least about 60 days after preparation, and an indefinite shelf life. This then permits development of sensors for detection of the broadest range of such toxic substances.

In a preferred embodiment, the sensor cells are engineered to express chimeric cell-surface receptors specific for a certain biochallenge (e.g., Staphylococcus aureus enterotoxin B). Those cells reside in a culture chamber that permits access of biochallenge agents to the culture medium, and thus to the sensor cells. Upon binding of the biochallenge with the chimeric receptor, the receptor undergoes a conformational change, which is recognized by certain of the sensor cell's native biomolecules, and initiates a cell activation response in that sensor cell. Such an activation response results in a characteristic entry of calcium ions to the cytoplasm from extracellular and/or intracellular calcium stores or pools. Those calcium ions will then diffuse to- and subsequently bind to the calcium-selective luminescent reporter present and/or expressed within the sensor cell (e.g., aequorin, obelin, et cetera), resulting in the emission of a calcium-dependent photonic signal having a characteristic spectral, spatial and temporal signature. Such a spectral signal may be characteristic of the calcium-dependent reporter molecule (e.g., an isoform of aequorin, et cetera) or of a fluorescent reporter specific for calcium ions (e.g., fura2) or of a suitable fluorescence energy transfer (FRET) reaction (e.g., with green fluorescent protein).

In initial embodiments, cells are labeled with their annunciator molecules by facilitated entries (microinjection through imbibing cells in solutions of permanent annunciators). In other embodiments, genetically engineered cells will express chimeric receptors selective for the agent of interest. In further embodiments, sensor cells will be derived from organisms that express such chimeric receptors as stable transformed strains of the host organism from which the sensor cells are derived. It is envisioned that in all such cases cells will express signals indicating changes in free cytosolic calcium, free radical, proton or other indicator through the use of reporters selective for those indicators; such may involve the creation of cell lines genetically engineered to express annunciators and constructs incorporating specific annunciators (e.g., aequorin, green fluorescent protein.)

All references recited herein are hereby incorporated in their entireties by reference as if each one were so referenced individually.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

What is claimed is:

1. A method for detecting a Foreign Substance in a fluid, comprising contacting a first macrophage having a reporter system incorporated therein with said fluid.

2. The method of claim 1, wherein said first macrophage recognizes a particular Foreign Substance.

3. The method of claim 2, wherein said Foreign Substance is selected from the group consisting of a bacterium, a pollen, a virus, a prion, a metabolic toxin, and a bioactive small molecule.

4. The method of claim 3, wherein said Foreign Substance is a pathogen or a toxin.

5. The method of claim 1, wherein said fluid is ambient air.

6. The method of claim 1, wherein the reporter system is luminescent or fluorescent.

7. The method of claim 6, wherein the reporter system includes use of aequorin, obelin, or fura2.

6. The method of claim 1, further comprising contacting a second macrophage having a reporter system incorporated therein with said fluid, wherein said first and said second macrophages each recognize a different particular Foreign Substance.

7. The method of claim 8, wherein said Foreign Substance is selected from one or more of the group consisting of a bacterium, a pollen, a virus, a prion, a metabolic toxin, and a bioactive small molecule.

8. The method of claim 8, wherein the first or second macrophage is derived from a poikilothermic organism.

9. The method of claim 1, further comprising an electronic means for detecting a reaction between said macrophage and said Foreign Substance.

10. The method of claim 8, further comprising an electronic means for detecting a reaction between said first or second macrophage and said Foreign Substances.

11. The use of a macrophage having a reporter system incorporated therein for detecting a Foreign Substance.

12. The use of a macrophage as set forth in claim 13, wherein the macrophage is derived from a poikilothermic cell.

13. The use of a macrophage as set forth in claim 14, wherein the reporter system is luminescent or fluorescent.

14. The use of a macrophage as set forth in claim 15, wherein the reporter system includes use of aequorin, obelin, or fura2.

15. The use of a macrophage as set forth in claim 13, wherein the macrophage is conventional or stationary.

16. A macrophage having a reporter system incorporated therein.

17. The macrophage of claim 18, wherein the reporter system is luminescent or fluorescent.

18. The macrophage of claim 19, wherein the reporter system includes use of aequorin, obelin, or fura2.

19. The macrophage of claim 18, wherein the macrophage is conventional or stationary.

20. The macrophage of claim 18, wherein the macrophage is derived from a poikilothermic animal.
